(19) United States
(12) Patent Application Publication
(10) Pub. No.: US 2026/0284113 A1
(43) Pub. Date: Sep. 24, 2026

(54) COMPOSITIONS AND METHODS FOR TREATING PERIPHERAL VASCULAR DISEASE

(71) Applicant: MAYO FOUNDATION FOR MEDICAL EDUCATION AND RESEARCH, Rochester, MN (US)

(72) Inventor: Atta Behfar, Rochester, MN (US)

(21) Appl. No.: 18/997,982

(22) PCT Filed: Jul. 25, 2023
(86) PCT No.: PCT/US2023/028598

§ 371 Date: Jan. 23, 2025

Related U.S. Application Data
(60) Provisional application No. 63/392,335, filed on Jul. 26, 2022.

(51) Int. Cl. A61K 35/19; A61K 9/00; A61K 9/06; A61P 9/10

(52) U.S. Cl. CPC A61K 35/19; A61K 9/0019; A61K 9/06; A61P 9/10

(57) ABSTRACT

A method of treating peripheral vascular disease, a vascular defect, or vascular dysfunction in a subject generally includes administering to the subject a therapeutic composition that includes a purified exosome product (PEP) and a pharmaceutically acceptable carrier. In one or more embodiments, the therapeutic composition further includes a supportive matrix.

### CROSS-REFERENCE TO RELATED APPLICATIONS

This application claims the benefit of U.S. Provisional Application Ser. No. 63/392,335, filed on Jul. 26, 2022, which is incorporated by reference herein in its entirety.

### SUMMARY

This disclosure describes, in one aspect, a method of treating peripheral vascular disease, a vascular defect, or vascular dysfunction in a subject. Generally, the method includes administering to the subject a therapeutic composition that includes a purified exosome product (PEP) and a pharmaceutically acceptable carrier.

In one or more embodiments, the PEP includes from 1% to 20% CD63⁻ exosomes and from 80% to 99% CD63⁺ exosomes.

In one or more embodiments, the PEP includes at least 50% CD63⁻ exosomes.

In one or more embodiments, the therapeutic composition further comprises a supportive matrix.

In one or more embodiments, the therapeutic composition further comprises a tissue sealant, fibrin glue, or a hydrogel.

In one or more embodiments, the therapeutic composition is applied in an amount effective to increase pro-angiogenic activity compared to peripheral vascular disease treated without the therapeutic composition.

In one or more embodiments, the therapeutic composition is applied in an amount effective to increase perfusion in vivo following ischemia compared to peripheral vascular disease treated without the therapeutic composition.

In one or more embodiments, the therapeutic composition is applied in an amount effective to increase drive of MAPK pathway or AKT pathway compared to peripheral vascular disease treated without the therapeutic composition.

In one or more embodiments, the therapeutic composition is delivered by intramuscular injection.

In one or more embodiments, the peripheral vascular disease, a vascular defect, or vascular dysfunction comprises peripheral arterial disease.

In one or more embodiments, the peripheral vascular disease, a vascular defect, or vascular dysfunction comprises atherosclerosis, ischemia, deep vein thrombosis, pulmonary embolism, varicose veins, chromic venous insufficiency, Buerger disease, Reynaud phenomenon, thrombophlebitis, or an aneurysm.

In one or more embodiments, the subject is a human. In one or more embodiments, the subject has an ischemic wound, and wherein the method increases ischemic wound closure as compared to a method not including PEP.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. PEP stimulates pro-angiogenic events in vitro. (A) Representative atomic force microscope (AFM) image of PEP exosomes. (B) Size distribution of PEP exosomes as measured by NANOSIGHT tracking analysis (Malvern Panalytical Ltd., Malvern, UK). (C) Zeta potential of exosomes measured using ZETASIZER instrument (Malvern Panalytical Ltd., Malvern, UK). Data presented as mean±S.D. (D) Stiffness of exosomes measured using AFM indentation. Young's modulus was used to express particle stiffness. Data presented as mean±S.D. (E) Western blotting analysis of exosomal marker proteins in fractionated samples. 10 μg of exosome protein was loaded per gel lane. Three separate CGMP batches of PEP were analyzed. (F) Quantification of INCUCYTE proliferation assay (Essen Bioscience, Inc. Ann Arbor, MI) using the INCUCYTE red channel to calculate percentage of red nuclei. (G) Scratch assay testing migration of HUVECs treated with FBS, PEP or serum free. Quantification of wound closure.

FIG. 2. PEP stimulates pro-angiogenic events in vitro. (A) INCUCYTE proliferation assay (Essen Bioscience, Inc. Ann Arbor, MI). Representative fluorescent images of FBS vs. PEP vs. serum free medium at 72 hours. (B) Representative fluorescent images of tube formation of HUVECs on Matrigel treated with PEP, VEGF, or suramin.

FIG. 3. PEP stimulates pro-angiogenic events in vitro. Scratch assay testing migration of HUVECs treated with FBS, PEP, or serum free medium. Representative pictures of proliferation for PEP vs. FBS vs. PBS (serum free) at 72 hours.

FIG. 4. PEP stimulates pro-angiogenic events in vitro. Tube formation of human umbilical vein endothelial cells (HUVECs) on MATRIGEL (Corning Life Sciences, Inc., Corning, NY) treated with PEP, VEGF, or suramin. (A) Quantification of tube length performed one hours, three hours, and six hours post-treatment. (B) Quantification of tube junction performed one hours, three hours, and six hours post-treatment.

FIG. 5. PEP stimulates pro-angiogenic events in vitro. Expression levels of 55 angiogenesis related proteins in human umbilical vein endothelial cells (HUVECs) was quantified at time 0, six hours, 12 hours, and 24 hours post PEP treatment. (n=4 per time point).

FIG. 6. Administration of PEP biogel promotes rat hind-limb perfusion three weeks post-ischemia. (A) Scheme of TISSEEL-PEP treatment for hind-limb ischemia in rats. (B) SEM imaging analysis of PEP biopotentiated TISSEEL (B, insert) TISSEEL without PEP. Exosomes are noted with arrow heads.

FIG. 7. Representative SPY (Stryker Corp., Kalamazoo, MI) angiography images of rats treated with fibrin glue alone (TISSEEL, Baxter International, Inc., Deerfield, IL), fibrin glue with PEP (TISSEEL-PEP), or negative control (sham) pre-op, post-op Day 0, and post-op Day 21.

FIG. 8. Quantification of global blood perfusion in rats treated with fibrin glue alone (TISSEEL, Baxter International, Inc., Deerfield, IL), fibrin glue with PEP (TISSEEL-PEP), or negative control (sham) pre-op (Pre-lig), post-op Day 0 (Post-lig D0), and post-op Day 21 (Post-lig D21). ****=p<0.0001.

FIG. 9. Quantification of blood perfusion in rats treated with fibrin glue alone (TISSEEL, Baxter International, Inc., Deerfield, IL), fibrin glue with PEP (TISSEEL-PEP), or negative control (sham). (A) Proximal limb section; (B) Middle limb section; (C) Distal Limb section. *=p<0.05; ***=p<0.005; ns.=non-significant.

FIG. 10. Immunofluorescence analysis of rat hind limb tissue in cross section. Rats were treated with fibrin glue alone (TISSEEL, Baxter International, Inc., Deerfield, IL), fibrin glue with PEP (TISSEEL-PEP), or negative control (sham), then stained for von Willebrand factor (vWF), smooth muscle actin (SMA), and proliferative marker 5-ethynyl-2′-deoxyuridine (EDU). Scale bar: 200 μm.

FIG. 11. Quantitation of vWF⁺, SMA⁺, and EdU⁺ cells in tissue sections shown in FIG. 10. (A) vWF; (B) SMA; (C) EdU. *=p<0.05 ; **=p<0.01; ****=p<0.0001.

FIG. 12. Quantitation of vascular area and fibrotic area in tissue sections. (A) Hematoxylin and eosin staining of tissue sections collected from rat hind limb tissues. (B) Mason's trichrome staining of tissue sections collected from rat hind limb tissues. (C) Vascular area calculated from the positive staining in the hematoxylin and eosin-stained sections. (D) Fibrotic area calculated from the Masson's trichrome stained sections. *=p<0.05; ***=p<0.005; ****=p<0.0001; ns.=non-significant.

FIG. 13. Expression analysis of genes involved in vascularization processes. (A) Heatmap of the expression of 84 angiogenesis related genes in the TISSEEL-PEP group and the TISSEEL group (n=3 per group). (B) Volcano plot of genes differentially regulated between TISSEEL-PEP and TISSEEL (control). (C) Heatmap of differentially regulated genes involved in endothelial cell proliferation. (D) Heatmap of differentially regulated genes involved in VEGFR signaling pathway.

FIG. 14. TISSEEL-PEP biogel improved blood perfusion in ischemic wound tissue. (A) Representative wound images at Day 0 and at Day 28 from tissue untreated (sham), treated with fibrin glue (TISSEEL, Baxter International Inc., Deerfield, IL) alone, or fibrin glue plus PEP (TISSEEL-PEP). (B) Quantification of wound healing area. Scale bar: 20 μm. **=p<0.01; ****=p<0.0001.

FIG. 15. TISSEEL-PEP biogel improved blood perfusion in ischemic wound tissue. (A) Representative SPY (Stryker Corp., Kalamazoo, MI) angiography images of ear wounds four weeks post-op for wounds untreated (sham), treated with fibrin glue (TISSEEL, Baxter International Inc., Deerfield, IL) alone, or fibrin glue plus PEP (TISSEEL-PEP). (B) Global blood perfusion quantified pre-op, post-op, and four weeks post-op. (C) Blood flow level in distal ear quantified. (D) Blood flow level in middle ear quantified. (E) Blood flow level in proximal ear quantified.

FIG. 16. Immunofluorescence images staining for CD31 and smooth muscle actin (SMA) in wounds treated with fibrin glue (TISSEEL, Baxter International Inc., Deerfield, IL) alone, treated with fibrin glue plus PEP (TISSEEL-PEP), or untreated (sham). Scale bar: 1 μm.

FIG. 17. Quantification of immunofluorescence. (A) CD31, normalized to control. (B) SMA, normalized to control. **=p<0.01; ***=p<0.005; ns.=non-significant.

FIG. 18. Quantification of rabbit tissue vascular area percentage in sections stained with hematoxylin and eosin. *=p<0.05; ***=p<0.005.

### DETAILED DESCRIPTION OF ILLUSTRATIVE EMBODIMENTS

This disclosure describes compositions and methods for treating peripheral vascular disease, a vascular defect, or vascular dysfunction in a subject. Generally, the methods include administering to the subject a purified exosome product (PEP) in an amount to ameliorate at least one symptom or clinical sign of the peripheral vascular disease, a vascular defect, or vascular dysfunction.

PEP is fully characterized and methods for preparing PEP are described in International Patent Application No. PCT/US2018/065627 (published as International Publication No. WO 2019/118817), U.S. Patent Publication No. 2021/0169812A 1 , and U.S. Pat. No. 10,596,123, each of which is incorporated by reference herein in its entirety. Briefly, PEP is a purified exosome product prepared using a cryodesiccation step that produces a product having a structure that is distinct from exosomes prepared using conventional methods. For example, PEP typically has a spherical or spheroidal structure and an intact lipid bilayer rather than a crystalline structure that results from the reaggregation of lipids of the exosome lipid bilayer after exosomes are disrupted during conventional exosome preparation methods. The spherical or spheroid exosome structures generally have a diameter of no more than 300 nm. Typically, a PEP preparation contains spherical or spheroid exosome structures that have a relatively narrow size distribution. In some preparations, PEP includes spherical or spheroidal exosome structures with a mean diameter of about 110 nm±90 nm, with most of the exosome structures having a mean diameter of 110 nm±50 nm such as, for example, 110 nm±30 nm.

An unmodified PEP preparation—i.e., a PEP preparation whose character is unchanged by sorting or segregating populations of exosomes in the preparation—naturally includes a mixture of CD63⁺ and CD63⁻ exosomes. Because CD63⁻ exosomes can inhibit unrestrained cell growth, an unmodified PEP preparation that naturally includes CD63⁺ and CD63⁻ exosomes can both stimulate cell growth for wound repair and/or tissue regeneration and limit unrestrained cell growth.

Further, by sorting CD63⁻ exosomes, one can control the ratio of CD63⁺ exosomes to CD63⁻ exosomes in a PEP product by removing CD63⁺ exosomes from the naturally-isolated PEP preparation, then adding back a desired amount of CD63⁺ exosomes. In one or more embodiments, a PEP preparation can have only CD63⁻ exosomes.

In one or more embodiments, a PEP preparation includes both CD63⁺ exosomes and CD63⁻ exosomes. The ratio of CD63⁺ exosomes to CD63⁻ exosomes can vary depending, at least in part, on the quantity of cell growth desired in a particular application. For example, a CD63⁺/CD63⁻ exosome ratio provides desired cell growth induced by the CD63+ exosomes and inhibition of cell growth provided by the CD63⁻ exosomes achieved via cell-contact inhibition. In certain scenarios, such as in tissues including non-adherent cells (e.g., blood derived components), this ratio may be adjusted to provide an appropriate balance of cell growth or cell inhibition for the tissue being treated. Since cell-to-cell contact is not a cue in, for example, tissue with non-adherent cells, one may reduce the CD63⁺ exosome ratio to avoid uncontrolled cell growth. Conversely, if there is a desire to expand out a clonal population of cells, such as in allogeneic cell-based therapy or immunotherapy, one can increase the ratio of CD63⁺ exosomes to ensure that a large population of cells can be derived from a very small source.

Thus, in one or more embodiments, the ratio of CD63⁺ exosomes to CD63⁻ exosomes in a PEP preparation may be at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, or at least 16:1. In one or more embodiments, the ratio of CD63⁺ exosomes to CD63⁻ exosomes in a PEP preparation may be at most 15:1, at most 16:1, at most 17:1, at most 18:1, at most 19:1, at most 20:1, at most 25:1, or at most 30:1. For example, the ratio of CD63⁺ exosomes to CD63⁻ exosomes may be between 1:1 to 30:1, 2:1 to 20:1, 4:1 to 15:1, or 8:1 to 10:1. In one or more certain embodiments, the PEP product is formulated to contain a 9:1 ratio of CD63⁺ exosomes to CD63⁻ exosomes. In one or more certain embodiments, native PEP, e.g., PEP with an unmodified ratio of CD63⁺ exosomes to CD63⁻ exosomes may be used.

Production of purified exosome product (PEP) involves separating plasma from blood, isolating a solution of exosomes from separated plasma with filtration and centrifugation. PEP is fully characterized and methods for preparing PEP are described in International Patent Application No. PCT/US2018/065627 (published as International Publication No. WO 2019/118817), U.S. Patent Publication No. 2021/0169812A1 , and U.S. Pat. No. 10,596,123, each of which is incorporated by reference herein in its entirety. Peripheral arterial disease (PAD) is a significant cause of morbidity and mortality. Therapeutic angiogenesis using extracellular vesicles to rescue ischemic tissues has produced modest results. This disclosure describes an alternative approach. Here, a purified exosome product (PEP), which is platelet-derived and VEGFR2-enriched, was evaluated as angiogenic therapy for PAD. Activating a VEGFR2-governed program, PEP enhanced angiogenic events in vitro. In vivo, local delivery of PEP induced new blood vessel formation, thereby augmenting tissue perfusion in rat ischemic hind limb and rabbit ischemic ear wound models. The present work introduces an off-the-shelf, translation-ready exosome-based regenerative strategy to drive angiogenesis for the treatment of ischemic diseases.

While described herein in the context of peripheral arterial disease as a model form of peripheral vascular disease, the compositions and methods described herein can involve any form of peripheral vascular disease, vascular defect, or vascular dysfunction. Peripheral vascular disease is a blood circulation disorder that causes the blood vessels of the peripheral vasculature to narrow, block, or spasm. Peripheral vascular disease can occur in arteries or veins. Functional peripheral vascular disease typically involves narrowing of blood vessels in response to factors including, but not limited to, brain signals or temperature changes. The narrowing causes blood flow to decrease, but there is no physical damage to the structure blood vessel. Organic peripheral vascular disease involves changes in blood vessel structure such as, for example, inflammation, plaques, and/or tissue damage. Exemplary forms of peripheral vascular disease, vascular defects, and vascular dysfunctions treatable using the methods described herein include, but are not limited to, atherosclerosis, ischemia, deep vein thrombosis, pulmonary embolism, varicose veins, chromic venous insufficiency, Buerger disease, Reynaud phenomenon, thrombophlebitis, or aneurysms.

### PEP Vesicles Have Distinct Exosomal Markers and Biophysical Pproperties

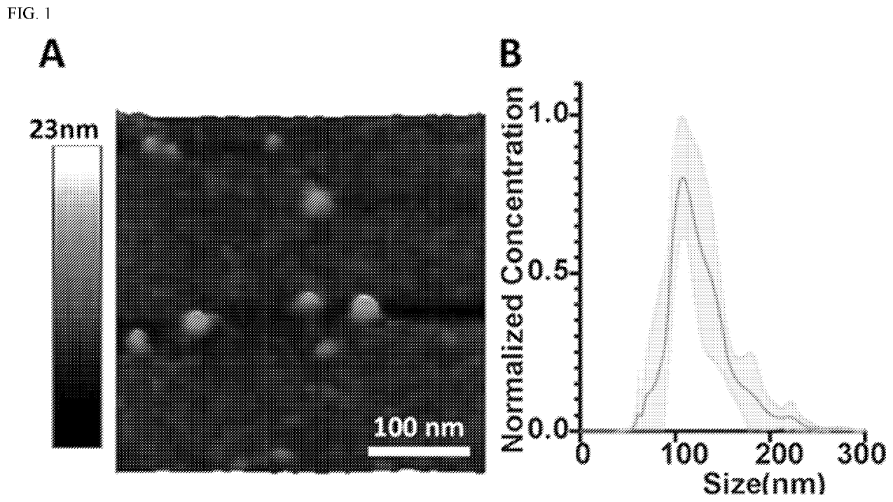

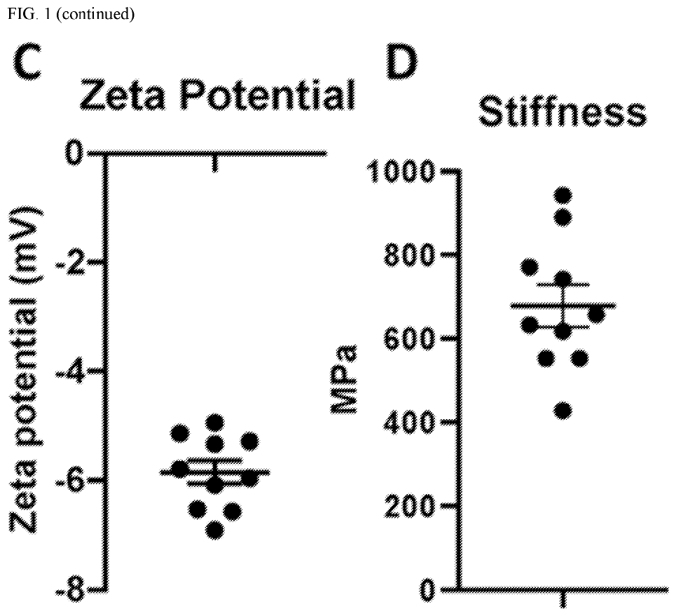

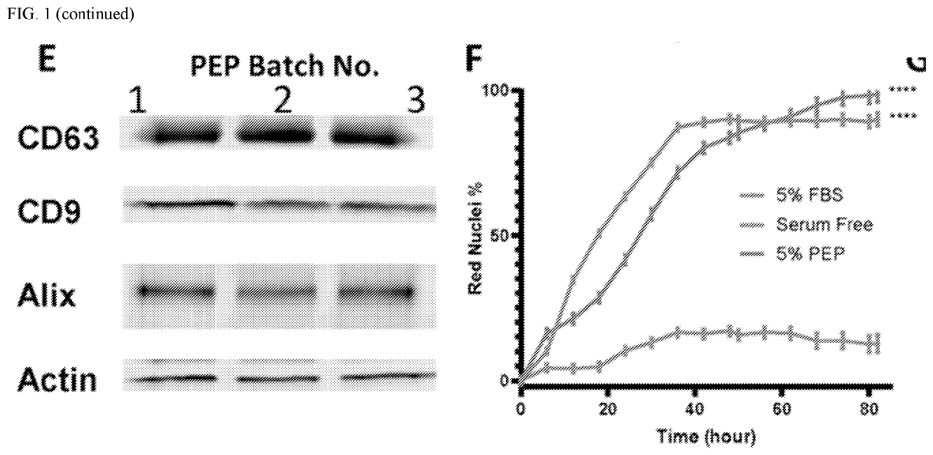

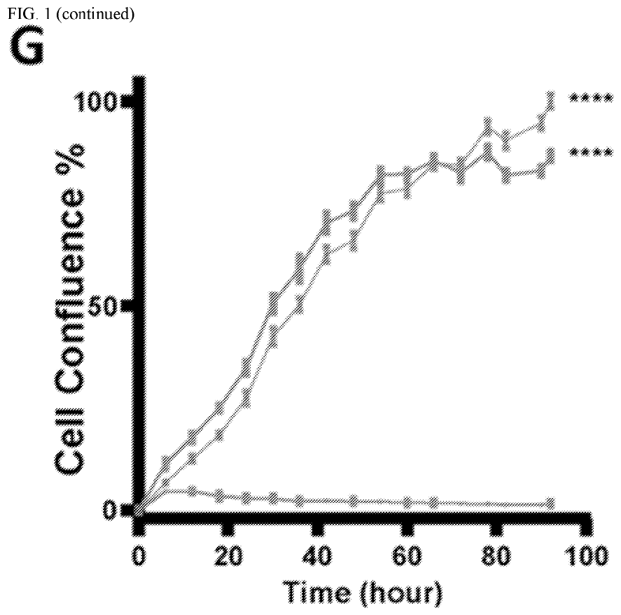

PEP was prepared and imaged by atomic force microscopy (AFM, FIG. 1A) to assess vesicle integrity and morphology and measured by nanoparticle tracking analysis (NTA) to assess the average particle size and concentration of the vesicle population (FIG. 1B). NTA demonstrated that the PEP preparation had a mean particle size of 126.7 nm and a mode size of 108.5 nm (FIG. 1B). Measured zeta potential, an average surface charge, revealed that all particles were negatively charged (−4.94 m V to −6.91 mV, FIG. 1C). PEP particles demonstrated a stiffness ranging from 428-890 MPa (FIG. 1D). Batch-to-batch consistency in the CGMP product was verified by cholesterol concentration, moisture level, and protein concentration, in tandem with consistent expression of CD63, CD9, and Alix (FIG. 1E). Human umbilical vein endothelial cells (HUVECs) exposed to PKH-labeled PEP, tracked through live-cell imaging, exhibited efficient vesicle uptake responsive to inhibitors of clathrin-mediated endocytosis, macropinocytosis or membrane fusion.

### PEP Enhances Pro-Angiogenic Cellular Activity in Vitro

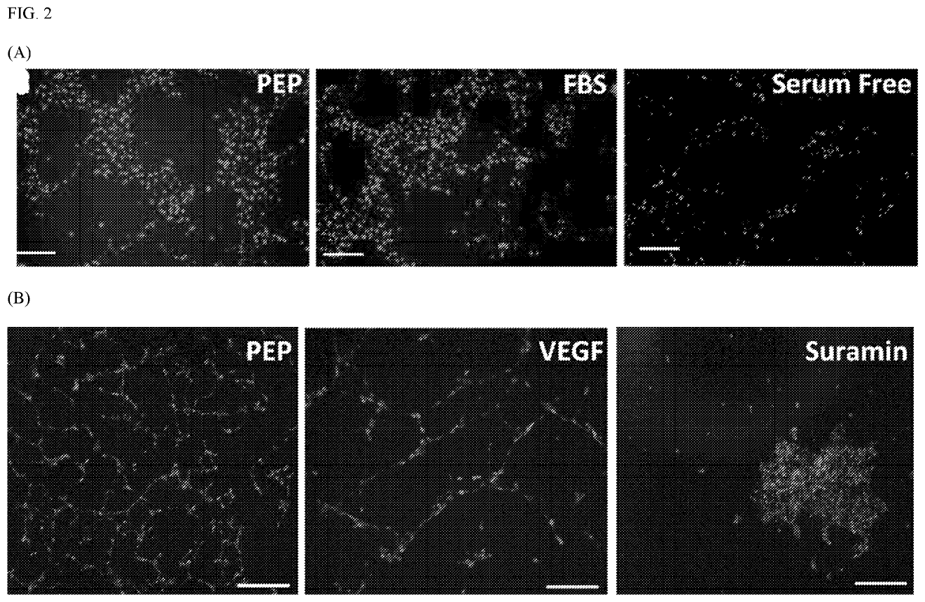

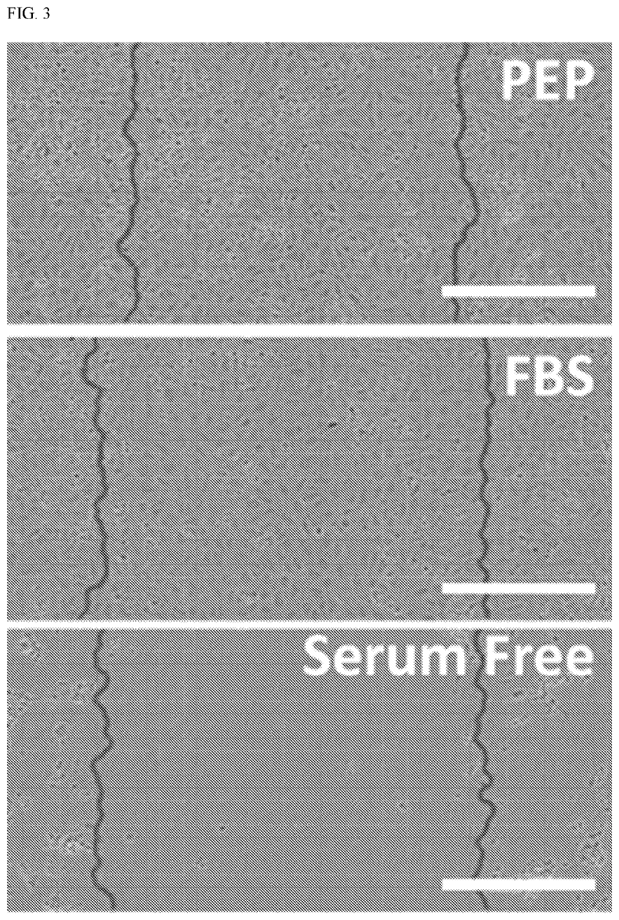

The effects of PEP on angiogenic events were tested by evaluating HUVEC proliferation, tube formation, and migration. A HUVEC proliferation assay was performed by culturing HUVECs with PEP using pre-defined optimal concentrations. Compared to FBS supplemented medium, PEP-treated (2.5×10¹¹ vesicles/mL) HUVECs achieved similar proliferative capacity (FIG. 1F, FIG. 2A), with higher expression of Ki-67. Angiogenic activity on a Matrigel substrate was performed using VEGF (100 ng/ml), suramin (100 μM), or basal medium supplemented with PEP (2.5×10¹¹ vesicles/mL) for six hours (FIG. 2B). Scratch wound assay on a HUVEC monolayer showed that PEP treatment facilitated wound closure, with the percentage of cell confluency higher in the PEP treated group than in the control (FIG. 1G, FIG. 3). Quantification of total length of branches (3053.68 vs. 2459.59 pixels, PEP vs. VEGF) and number of junctions (70.75 vs. 59.75, PEP vs. VEGF) per field were higher in the PEP-treated group (FIG. 2B, FIG. 4). HUVECs treated with 2.5×10¹¹ vesicles/mL, and screened using an angiogenesis antibody array, displayed a significant increase in proangiogenic paracrine factors 24 hours after treatment with PEP (FIG. 5), validating in vitro angiogenic activity induced by PEP.

### TISSEEL-PEP Biogel Promoted Angiogenesis of Hindlimb Ischemia

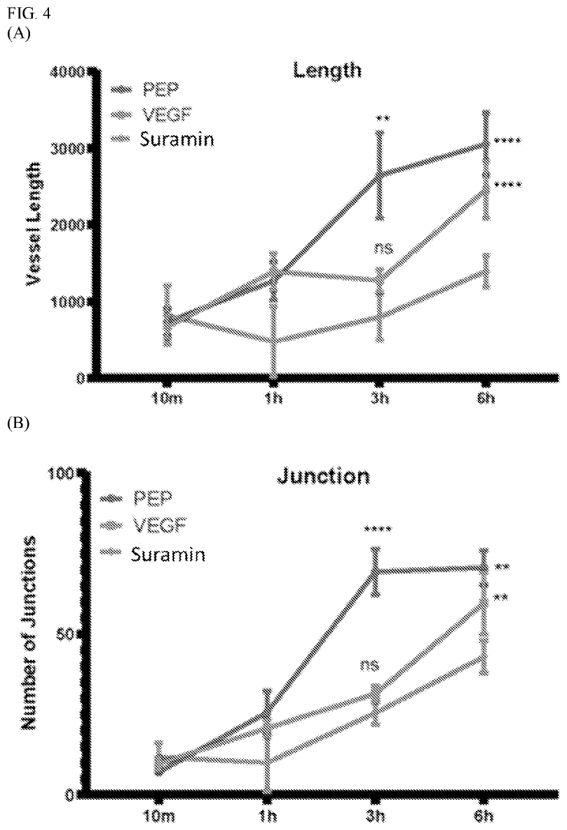

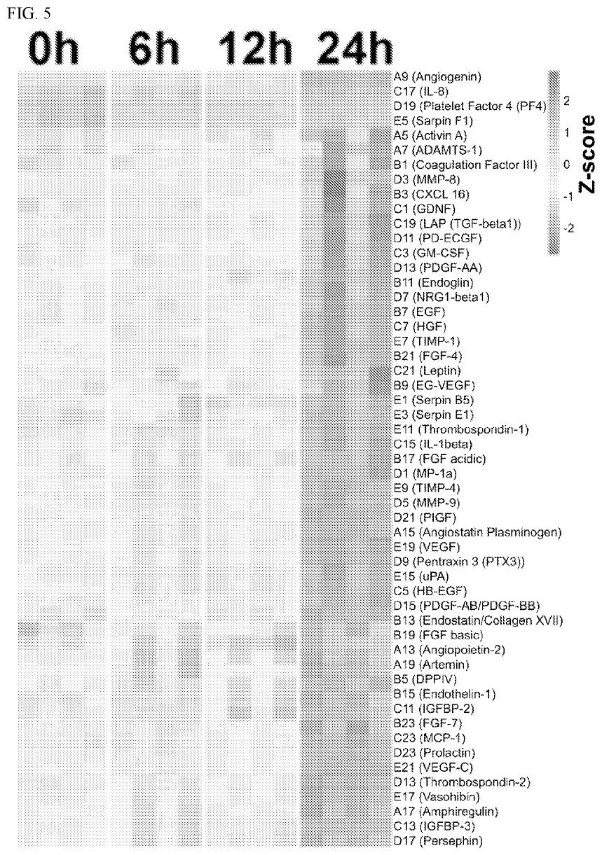

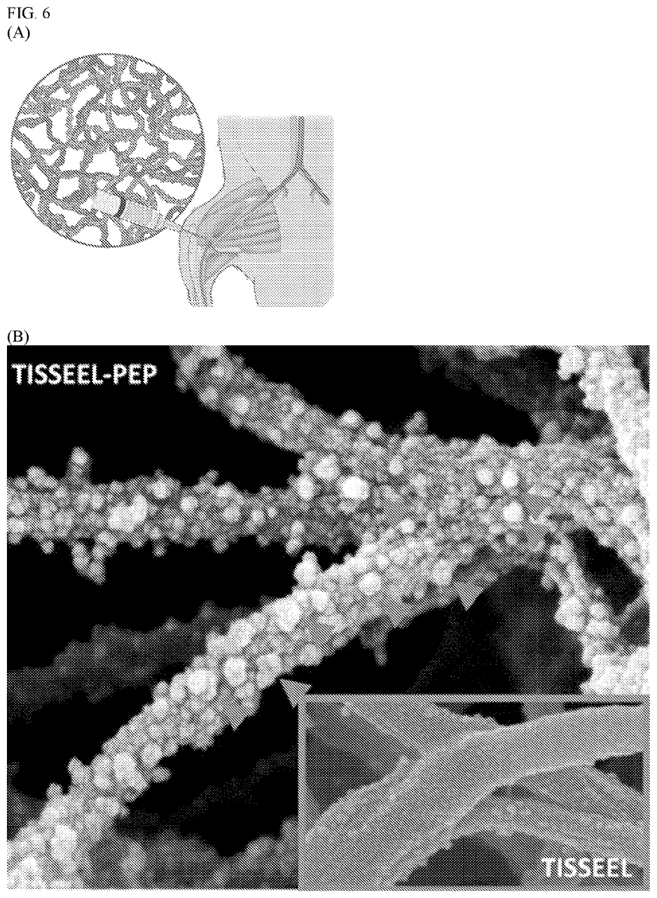

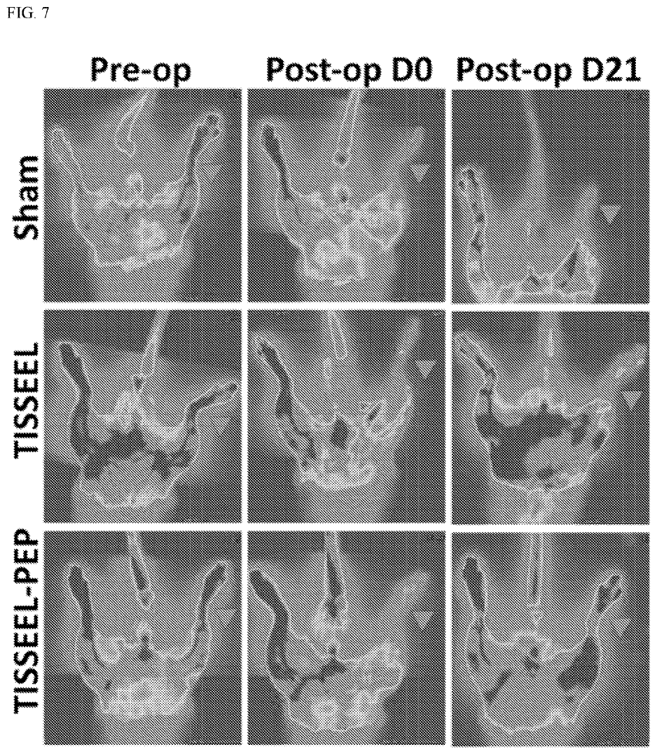

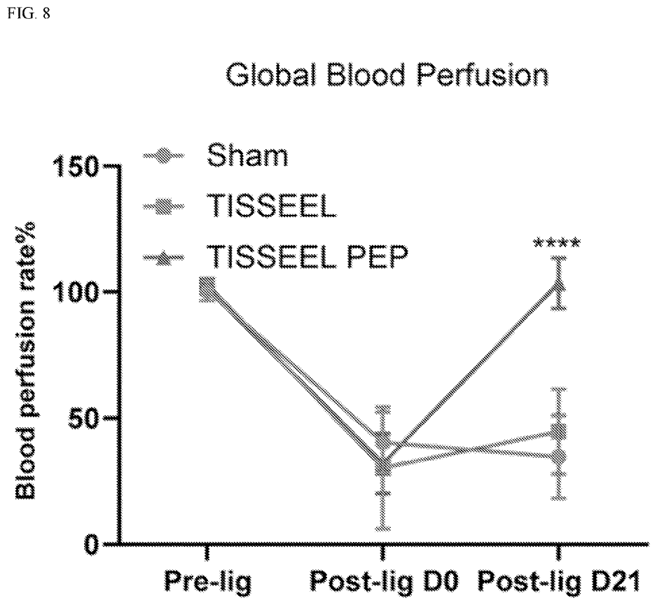

Crosslinking enables slow release of bioactive components, enhancing therapeutic potency. Here, PEP was incorporated into TISSEEL (Baxter International, Deerfield, IL), a clinical grade fibrin glue, to investigate whether sustained PEP release could rescue a model of hindlimb ischemia (HLI) (FIG. 6A). 1×10¹² PEP vesicles/mL provided consistent release of exosomes between 2×10¹¹ vesicles/mL and 3×10¹¹ vesicles/mL over a two-week follow-up. Following induction of hindlimb ischemia, rats were randomly assigned to saline, TISSEEL, or TISSEEL-PEP injection at the site of ligation. Perfusion was tracked pre-operatively, immediately post operation, and at day 21 post operation. Vessel occlusion halved perfusion to the ischemic limb, as measured by SPY angiography (FIG. 7). In rats treated with TISSEEL-PEP, perfusion was restored to the value of the non-ischemic control limb by day 21 post operation, while sham or TISSEEL-treated groups failed to substantially recover in distal, middle, and proximal limb regions (FIG. 8, FIG. 9).

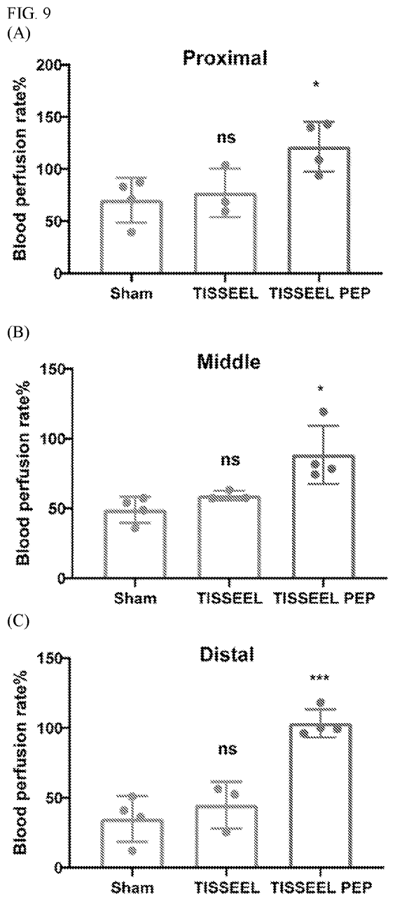

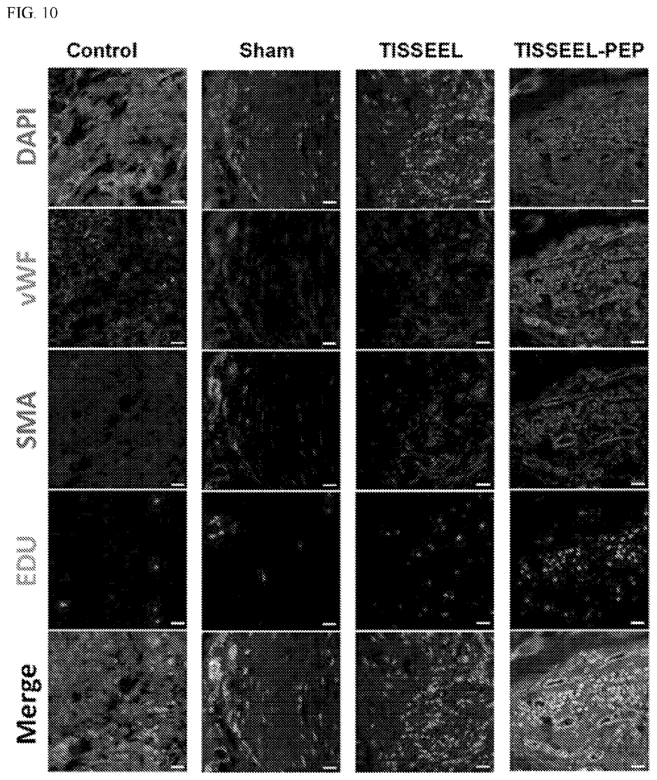

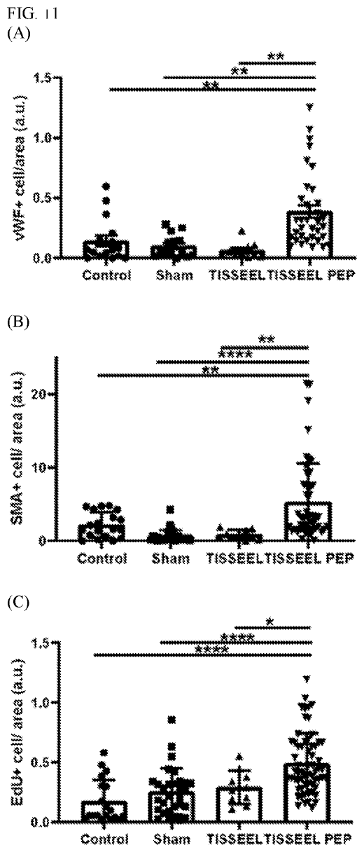

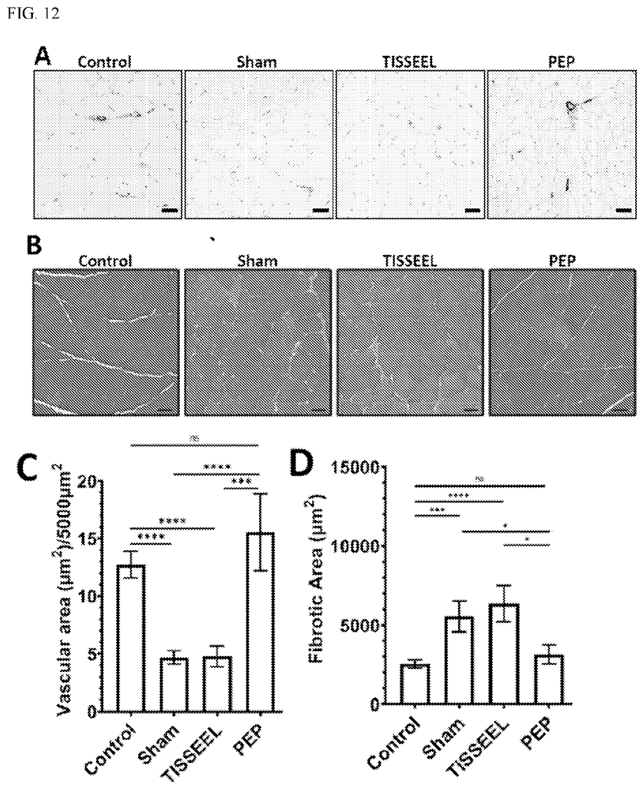

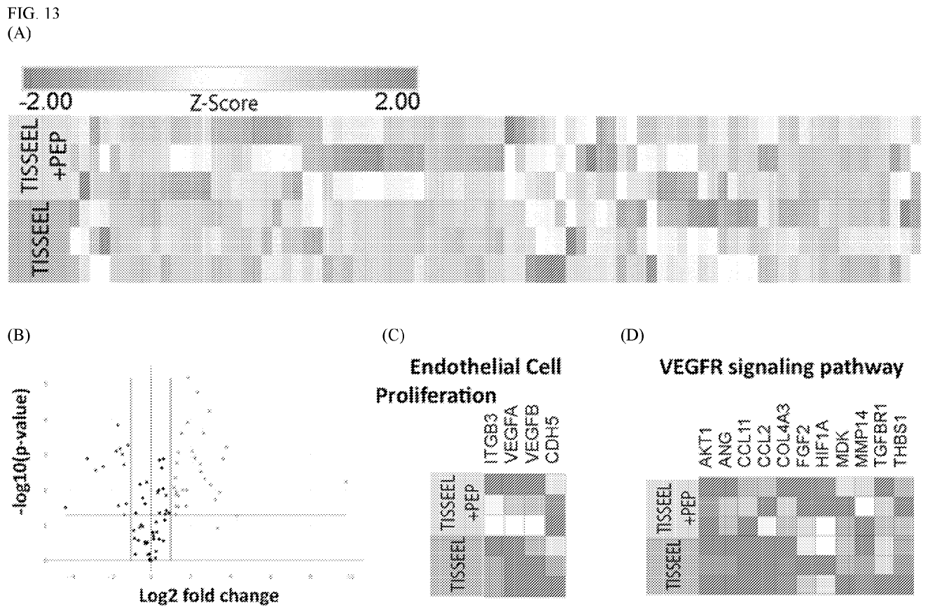

To assess angiogenic effects, vascular cells were immunohistochemically stained and quantified with the endothelial marker Von Willebrand factor (VWF), the smooth muscle marker smooth muscle actin (SMA) and the proliferative marker EdU (FIG. 10). All three were significantly higher in the TISSEEL-PEP group compared with sham or TISSEEL alone (FIG. 10, FIG. 11). Closer examination revealed substantial increase in vascularized area for the TISSEEL-PEP group only (FIG. 12A, C). Furthermore, sham or TISSEEL-treated rats had muscle fibers that appeared more fibrotic, with characteristic collagen distribution (FIG. 12B, D). In contrast, the TISSEEL-PEP group demonstrated level of collagen similar to non-ischemic tissue (FIG. 12B). Thus, treatment with TISSEEL-PEP biogel appeared to restore perfusion and rescue ischemic tissue damage. To address the mechanistic basis for PEP action, muscle samples obtained at Day 21 were profiled for expression of 84 angiogenesis related genes (FIG. 13A) and noted to significantly upregulate 29 (FIG. 13B). Pathway analysis suggested PEP-based activation of endothelial proliferation and VEGFR signaling events (FIG. 13C, D).

### TISSEEL-PEP Biogel Rescues Perfusion in Rabbit Ischemic Wound Model

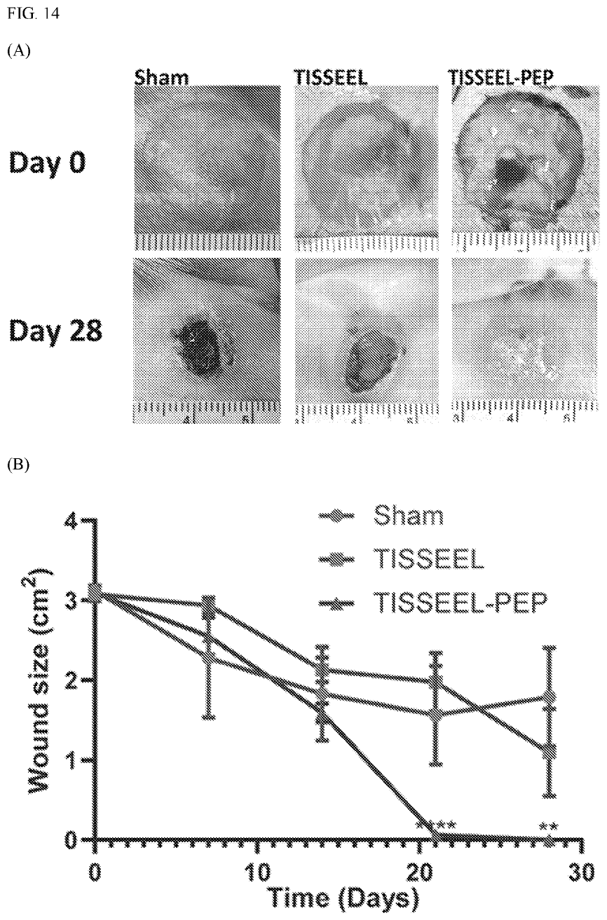

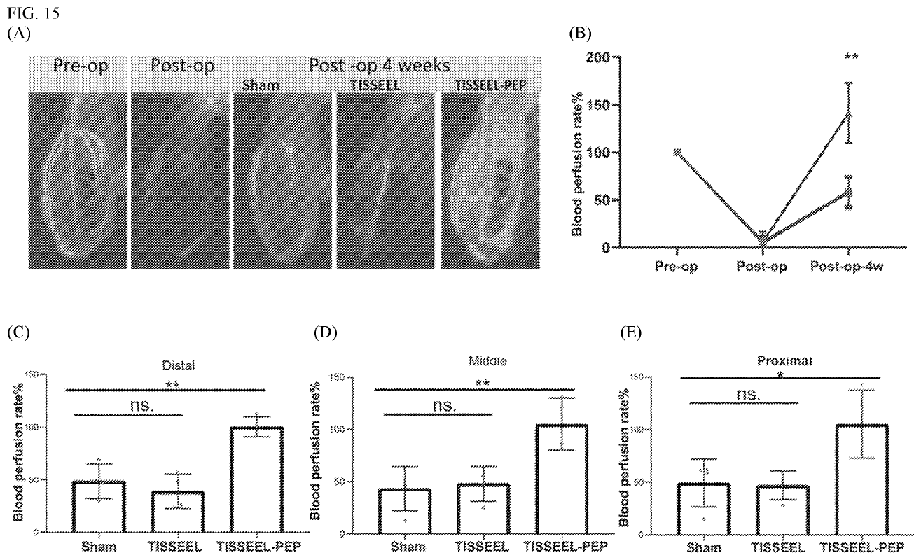

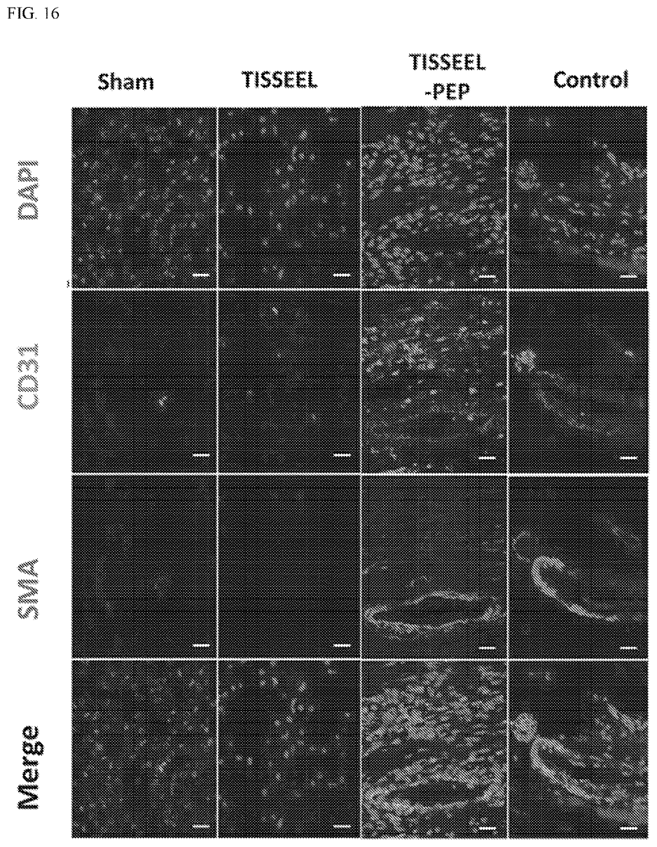

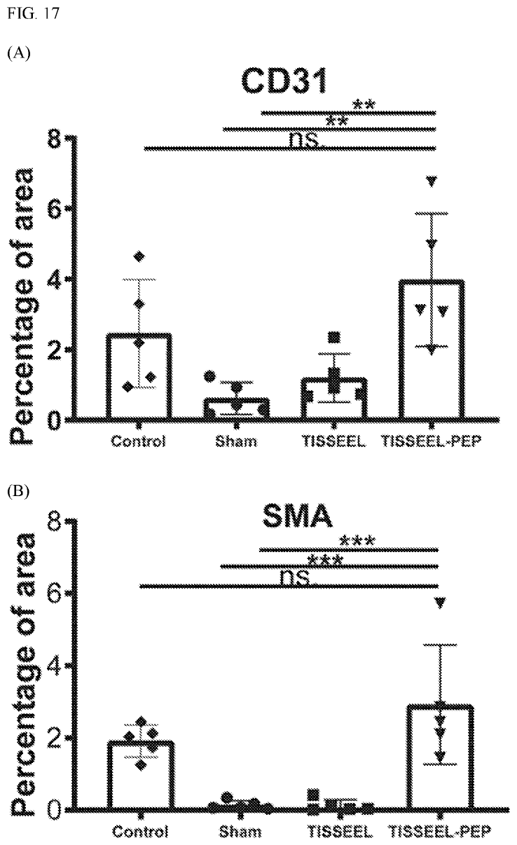

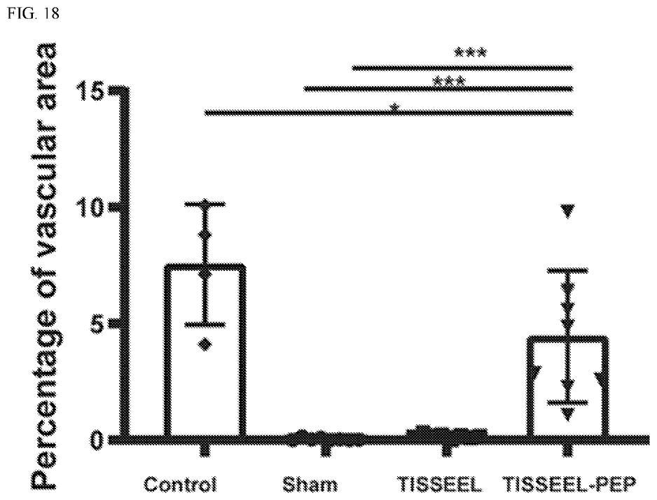

To further access the efficacy and translational value of TISSEEL-PEP biogel, the approach was tested in large animals, where perfusion deficits can lead to irreversible tissue necrosis and damage. A well-defined rabbit ear ischemic wound model was employed in which less than 20% blood supply is retained postoperatively, as previously described (Chien, S., Wound Rep Reg. 15:928-935, 2007). TISSEEL-PEP biogel was placed on top of the wound bed while uninjured, sham treated and TISSEEL-treated ears were used as baseline and treatment controls. Following four weeks of treatment, TISSEEL-PEP significantly improved ischemic wound closure versus sham and TISSEEL treatment alone (FIG. 14A, B). At four weeks, both SPY angiography and histological analysis revealed that TISSEEL-PEP treatment group restored blood flow to distal, middle, and proximal sections of ears, while controls failed to resuscitate blood flow (FIG. 15). Immunohistochemical staining indicated no angiogenesis in the sham or TISSEEL arms compared with healthy controls, while a significant increase in vessel formation in the TISSEEL-PEP group was documented by increases in CD31 and SMA expression (FIG. 16, FIG. 17). Histological analysis of hematoxylin and eosin (HE)-stained tissues quantified augmented vascularity in TISSEEL-PEP treated cohorts (FIG. 18). Taken together, these results validated the angiogenic impact of PEP in a large animal model of ischemic injury.

This disclosure therefore describes the mechanism by which PEP exosomes drive pro-angiogenic events through pVEGFR-2 donation. PEP triggered endothelial cell proliferation, migration, and vascular tube formation, with protein profiling at 24 hours post-treatment documenting vasculogenic polarization. MAPK and AKT were activated in intracellular pathways, revealing that PEP donates bioactive pVEGFR-2, precluding the need for a growth factor rich environment. In corporation PEP into a fibrin-glue-based composition mediated sustained release of PEP, resulted in proangiogenic cellular events and enhanced blood vessel formation in distinct models of peripheral vascular occlusion.

Ischemic diseases are prevalent worldwide, and few treatments exist to restore blood flow beyond procedural revascularization. Platelets are established drivers for angiogenic events following tissue injury. PEP, purified exosomes derived from activated platelets, provide a platform by which to evaluate the mechanistic basis for blood vessel formation and for treating peripheral vascular diseases.

VEGFR-2 signaling is a well-characterized pathway, crucial in cellular processes that underpin blood vessel formation. During angiogenesis, phosphorylated VEGFR-2 activates multiple downstream pathways via signaling intermediates including MAPK, AKT, and GTPases. Beyond cell proliferation, migration and tube formation, VEGFR-2 also modulates vessel permeability and potent survival factors. Initial efforts to develop pro-angiogenic therapies focused on direct delivery of growth factors (GFs) into areas of ischemia. However, a short-half life and the initial burst-release profile were associated with limited efficacy and unmasked adverse effects associated with treatment with these growth factors. With advancements in material science, different biomatrices including fibrin, alginate, and hyaluronic hydrogels have been used as delivery vehicles demonstrating local, sustained, and degradable capacities in angiogenesis studies.

This study establishes exosomes as capable of delivering bioactive proteins to drive angiogenic events. PEP-mediated delivery of pVEGFR-2 initiated a fine-tuned signaling network in endothelial cells relevant to angiogenesis both in vitro and in vivo. In PEP-treated HUVECs, proangiogenic cellular activity was observed. Moreover, increases of VEGFR-2 expression resulted in rapid induction of downstream MAPK and AKT pathways, pointing to protein donation rather than transcription and translation as a new modality capable of driving these events. Following growth factor stimulation, VEGFR-2 is internalized from the cell surface via a clathrin-mediated and dynamin-mediated endocytosis, micropinocytosis, and membrane fusion. PEP was here found to mimic this process in a growth-factor-independent-independent fashion with cellular internalization found suppressed by inhibitors of these pathways.

PEP biopotentiation of a fibrin-based biogel (TISSEEL, Baxter International, Deerfield, IL) achieved controlled release of exosomes over a sustained period to drive targeted biological events. Although unachievable with recombinant proteins due to rapid deterioration, purification of exosomes with an intact lipid bilayer here allowed stability and secured compatibility with sustained release strategies. Leveraging this platform, the present study assessed benefit in a small animal rodent model, further validated in a large animal rabbit model to demonstrate cross-species viability of observed findings and to secure sufficient pre-clinical evidence for clinical translation.

This disclosure therefore describes compositions and methods for treating peripheral vascular disease in a subject. Generally, the compositions include PEP and a pharmaceutically acceptable carrier. In a surgical setting, the PEP may be combined with a suitable carrier such as, for example, a surgical glue, a tissue adhesive, and/or a supportive matrix (e.g., a collagen scaffold).

Thus, the method includes administering an effective amount of the composition to subject. In this aspect, an “effective amount” is an amount effective to ameliorate (e.g., improve at least partially) at least one symptom or clinical sign of peripheral vascular disease, a vascular defect, or vascular dysfunction. As used herein, the term “symptom” refers to any subjective evidence of disease or of a patient's condition, while the terms “sign” or “clinical sign” refers to an objective physical finding relating to a particular condition capable of being found by one other than the patient. Thus, for example, the method can include administering the composition to the subject in an amount effective to enhance pro-angiogenic activity compared to a subject treated comparably (e.g., treatment with or without a suitable carrier as described in more detail below) but without PEP, to increase perfusion in vivo following ischemia compared to a subject treated comparably but without PEP, and/or to increase drive of MAPK and/or AKT pathways compared to a subject treated comparably but without PEP.

Exemplary indicators of enhanced pro-angiogenic cellular activity include, but are not limited to, increased cell proliferation, increased tube formation (e.g., increase branch length, increase number of junctions, etc.), increased cell migration (e.g., decreased time to cell confluency in vitro or in vivo), or increased presence of pro-angiogenic factors. In all cases, the specific indicator of enhanced pro-angiogenic activity is in comparison to a subject treated comparably but without PEP.

Exemplary indicators of increased perfusion in vivo following ischemia include, but are not limited to, decreased time to perfusion (e.g., of proximal, middle, and/or distal regions), increased expression of endothelial markers (e.g., Von Willebrand factor, smooth muscle actin, 5-ethynyl-2′-deoxyuridine (EdU)), increased areas of vascularization, and/or decreased extent of fibrosis. In all cases, the specific indicator of increased perfusion in vivo following ischemia is in comparison to a subject treated comparably but without PEP.

As used herein, a “subject” can be a human or any non-human animal. Exemplary non-human animal subjects include, but are not limited to, a livestock animal or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

PEP may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, hydrogel, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained. As noted above, in a surgical setting, exemplary suitable carriers include surgical glue, tissue adhesive, or a supportive matrix (e.g., a collagen scaffold).

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, etc.), or topical (e.g., application to nervous tissue exposed during surgery, intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release. Thus, for example, in one or more embodiments, the pharmaceutical composition may be formulated for intramuscular injection, intravenous administration, or subcutaneous administration.

Thus, a pharmaceutical composition may be provided in any suitable form including but not limited to a solution, a suspension, an emulsion, a spray, an aerosol, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both, and then, if necessary, shaping the product into the desired formulations.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In one or more embodiments, a dose of PEP can be measured in terms of the PEP exosomes delivered in a dose. Thus, in one or more embodiments, the method can include administering sufficient PEP to provide a dose of, for example, from about 1×10⁶ PEP exosomes to about 1×10¹⁵ PEP exosomes to the subject, although in one or more embodiments the methods may be performed by administering PEP in a dose outside this range.

In one or more embodiments, therefore, the method can include administering sufficient PEP to provide a minimum dose of at least 1×10⁶ PEP exosomes, at least 1×10⁷ PEP exosomes, at least 1×10⁸ PEP exosomes, at least 1×10⁹ PEP exosomes, at least 1×10¹⁰ PEP exosomes, at least 1×10¹¹ PEP exosomes, at least 2×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 8×10¹¹ PEP exosomes, at least 9×10¹¹ PEP exosomes, at least 1×10¹² PEP exosomes, 2×10¹² PEP exosomes, at least 3×10¹² PEP exosomes, at least 4×10¹² PEP exosomes, or at least 5×10¹² PEP exosomes, at least 1×10¹³ PEP exosomes, or at least 1×10¹⁴ PEP exosomes.

In one or more embodiments, the method can include administering sufficient PEP to provide a maximum dose of no more than 1×10¹⁵ PEP exosomes, no more than 1×10¹⁴ PEP exosomes, no more than 1×10¹³ PEP exosomes, no more than 1×10¹² PEP exosomes, no more than 1×10¹¹ PEP exosomes, or no more than 1×10¹⁰ PEP exosomes.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1×10¹¹ to 1×10¹³ PEP exosomes such as, for example, a dose of from 1×10¹¹ to 5×10¹² PEP exosomes, a dose of from 1×10¹² to 1×10¹³ PEP exosomes, or a dose of from 5×10¹² to 1×10¹³ PEP exosomes. In certain embodiments, the method can include administering sufficient PEP to provide a dose that is equal to any minimum dose or any maximum dose listed above. Thus, for example, the method can involve administering a dose of 1×10¹⁰ PEP exosomes, 1×10¹¹ PEP exosomes, 5×10¹¹ PEP exosomes, 1×10¹² PEP exosomes, 5×10¹² PEP exosomes, 1×10¹³ PEP exosomes, or 1×10¹⁴ PEP exosomes.

Alternatively, a dose of PEP can be measured in terms of the concentration of PEP upon reconstitution from a lyophilized state. Thus, in one or more embodiments, the methods can include administering PEP to a subject at a dose of, for example, from about a 0.01% solution to a 100% solution to the subject, although in one or more embodiments the methods may be performed by administering PEP in a dose outside this range. As used herein, a 100% solution of PEP refers to one vial of PEP (approximately 2×10¹¹ exosomes or 75 mg) solubilized in 1 mL of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell conditioned media.

In one or more embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In one or more embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1% to 50% such as, for example, a dose of from 5% to 20%. In certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

A single dose may be administered as one administration, continuously for a prescribed period of time, or in multiple discrete administrations. When multiple administrations are used, the amount of each administration may be the same or different. For example, a prescribed daily dose of may be administered as a single dose, continuously over 24 hours, as two administrations, which may be equal or unequal. When multiple administrations are used to deliver a single dose, the interval between administrations may be the same or different. In certain embodiments, PEP may be administered as a once-off administration, for example, during a surgical procedure.

In certain embodiments in which multiple administrations of the PEP composition are administered to the subject, the PEP composition may be administered as needed to treat peripheral vascular disease to the desired degree. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In one or more embodiments, the method can include multiple administrations of PEP to a subject at an interval (for two administrations) or intervals (for more than two administrations) characterized by a range having endpoints defined by any minimum interval identified above and any maximum interval that is greater than the minimum interval. For example, in one or more embodiments, the method can include multiple administrations of PEP at an interval or intervals of from one day to six months such as, for example, from three days to ten days. In one or more embodiments, the method can include multiple administrations of PEP at an interval of that is equal to any minimum interval or any maximum interval listed above. Thus, for example, the method can involve multiple administrations of PEP at an interval of three days, five days, seven days, ten days, 14 days, 21 days, one month, two months, three months, or six months.

In one or more embodiments, the methods can include administering a cocktail of PEP that is prepared from a variety of cell types, each cell type having a unique pro-angiogenic, pro-perfusion recovery, or pro-MAPK/AKT driving profile—e.g., protein composition and/or gene expression. In this way, the PEP composition can provide a broader spectrum of activity needed to treat peripheral vascular disease than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXEMPLARY EMBODIMENTS

Embodiment 1 is a method of treating peripheral vascular disease, a vascular defect, or vascular dysfunction in a subject, the method including administering to the subject a therapeutic composition including a purified exosome product (PEP) and a pharmaceutically acceptable carrier.

Embodiment 2 is the method of embodiment 1, wherein the PEP includes spherical or spheroid exosomes having a diameter no greater than 300 nm.

Embodiment 3 is the method of embodiment 1, wherein the PEP includes spherical or spheroid exosomes having a mean diameter of 110 nm±90 nm.

Embodiment 4 is the method of embodiment 3, wherein the PEP includes spherical or spheroid exosomes having a mean diameter of 110 nm±50 nm.

Embodiment 5 is the method of embodiment 4, wherein the PEP includes spherical or spheroid exosomes having a mean diameter of 110 nm±30 nm.

Embodiment 6 is the method of any preceding embodiment, wherein the PEP includes from 1% to 20% CD63⁻ exosomes and from 80% to 99% CD63⁺ exosomes.

Embodiment 7 is the method of any one of embodiments 1-5, wherein the PEP includes at least 50% CD63⁻ exosomes.

Embodiment 8 is the method of any preceding embodiment, wherein the PEP includes from 1×10¹¹ PEP exosomes to 1×10¹³ PEP exosomes.

Embodiment 9 is the method of embodiment 8, wherein the PEP includes from 1×10¹² PEP exosomes to 1×10¹³ PEP exosomes.

Embodiment 10 is the method of any preceding embodiment, wherein the therapeutic composition further includes a supportive matrix.

Embodiment 11 is the method of embodiment 10, wherein the supportive matrix includes a collagen scaffold.

Embodiment 12 is the method of any preceding embodiment, wherein the therapeutic composition further includes a tissue sealant, fibrin glue, or a hydrogel.

Embodiment 13 is the method of any preceding embodiment, wherein the therapeutic composition is applied in an amount effective to increase pro-angiogenic activity compared to peripheral vascular disease treated without the therapeutic composition.

Embodiment 14 is the method of any preceding embodiment, wherein the therapeutic composition is applied in an amount effective to increase perfusion in vivo following ischemia compared to peripheral vascular disease treated without the therapeutic composition.

Embodiment 15 is the method of any preceding embodiment, wherein the therapeutic composition is applied in an amount effective to increase drive of the MAPK pathway or the AKT pathway compared to peripheral vascular disease treated without the therapeutic composition.

Embodiment 16 is the method of any preceding embodiment, wherein the therapeutic composition is delivered by intramuscular injection.

Embodiment 17 is the method of any preceding embodiment, wherein the peripheral vascular disease, a vascular defect, or vascular dysfunction includes peripheral arterial disease.

Embodiment 18 is the method of any preceding embodiment, wherein the peripheral vascular disease, a vascular defect, or vascular dysfunction includes atherosclerosis, ischemia, deep vein thrombosis, pulmonary embolism, varicose veins, chromic venous insufficiency, Buerger disease, Reynaud phenomenon, thrombophlebitis, or an aneurysm.

Embodiment 19 is the method of any preceding embodiment, wherein the subject is a human.

Embodiment 20 is the method of any preceding embodiment, wherein the subject has an ischemic wound, and wherein the method increases ischemic wound closure as compared to a method not including PEP.

### EXAMPLES

### Cell Culture

Human umbilical vascular endothelial cells (HUVECs, Lonza Group AG, Basel, Switzerland) and GFP-tagged HUVECs (Essen BioScience, Inc., Ann Arbor, MI) were cultured in a 37° C. humidified chamber with 5% CO₂ in EBM-2 endothelial cell growth basal medium (Lonza Group AG, Basel, Switzerland) using an EGM-2 BULLET kit (Lonza Group AG, Basel, Switzerland).

### Purified Exosome Product (PEP)

PEP (Rion LLC, Rochester, MN) was isolated by subjecting pooled platelets. First, thermal shock was utilized to activate platelets, verified as previously described (Kamath et al., 2001, Eur Heart 22:1561-1571). The derived product was then subjected to repeat enucleation, serial filtration, and staged centrifugation for elimination of non-exosome components.

Following an encapsulation step, PEP was derived as a dry powder through lyophilization with each lyophilized vial containing approximately 5×10¹² vesicles/mL. A 100% PEP solution was defined as dissolving one vial of lyophilized PEP in 1 mL of phosphate buffered saline (PBS) representing 5×10¹² vesicles/mL. Before use, the resuspended PEP solution was filtered using a 0.22-μm filter system (STERIFLIP, MilliporeSigma, Burlington, MA). For cell culture experiments, PEP was reconstituted in designated culture medium.

### TISSEEL-PEP Biogel Preparation and Scanning Electron Microscopy (SEM)

TISSEEL-PEP biogel was prepared by reconstituting PEP in the fibrinolysis inhibitor solution from TISSEEL fibrin glue preparation kit (Baxter International, Inc., Deerfield, IL). The TISSEEL preparation protocol was then followed according to the manufacturer's instructions.

For SEM, samples were fixed in Trumps fixative at 4° C. overnight, washed in PBS, rinsed in deionized H₂O, dehydrated, and critical point dried prior to imaging on a cold field emission scanning electron microscope at 5 kV accelerating voltage (S-4700, Hitachi High-Technologies Corp., Tokyo, Japan).

### Nanoparticle Tracking Analysis (NTA)

PEP samples were diluted in PBS. Nanoparticle tracking analysis of exosome size and particle number was performed using a NANOSIGHT NS300 system (Malvern Panalytical, Malvern, United Kingdom) following the manufacturer's instructions.

### Zeta Potential Measurement

PEP samples were diluted in PBS (Sigma) for zeta potential analysis using a Zetasizer Nanos Dynamic Light scattering (Malvern Panalytical, Malvern, United Kingdom). All experiments were performed at a constant temperature of 25° C., and carried out at the Matexcel Materials Analysis Laboratory, Bohemia, NY.

### Atomic Force Microscopy (AFM)

Diluted PEP samples were plated on freshly cleaved mica substrate (Ted Pella, Inc., Redding CA) for 20 minutes, washed three times with deionized H₂O, and gently dried with nitrogen gas stream. Images of 2 μm×2 μm (width×length) were collected by atomic force microscopy (NANOSCOPE IV PICOFORCE multimode atomic force microscope; Bruker Scientific Instruments, Inc., Billerica, MA) in contact mode at room temperature and analyzed by NANOSCOPE analysis software (Bruker Scientific Instruments, Inc., Billerica, MA).

### Stiffness Measurement

Particle stiffness test was performed as previously described (Zhang et al., 2018, Nat Cell Biol 20(3): 332-343). Freshly cleaved mica coverslips were coated with poly-L-lysine (0.1% wt/vol in H₂O) for 30 minutes, followed by incubation with the sample for 45 minutes. Samples were then rinsed three times with PBS buffer and submerged in PBS for measurements. All measurements were done on an atomic force microscope (MFP 3D, Oxford Instruments Asylum Research, Santa Barbara, CA). Cantilever spring constant was calibrated by the thermal method resulting in constants of 1.2-1.8 N/m. The radius of curvature of the cantilever was ~10 nm and the Hertz model was used to analyze force curves for stiffness determination. An array of force curves on each sample was measured with at least 10 data points collected per sample.

### LC-MS/MS Cholesterol Quantification

Bligh and Dyer extraction was performed on reconstituted PEP samples. The samples containing the analytes were injected with appropriate dilution for LC-MS/MS analysis through revered phase method, carried out in part at the lipid analysis laboratory in Avanti Polar Lipids, Inc. (Alabaster, AL).

### Moisture

The moisture content of PEP was determined using a moisture analyzer (MB90, Ohaus Corp., Parsippany, NJ). Samples were weighed, rapidly heated with a halogen dryer so the moisture vaporized, and then reweighed to determine percent moisture content.

### Protein Extraction and Quantification

HUVEC or PEP samples were homogenized in lysis buffer containing: 50 mM NaPyrophosphate, 50 mM NaF, 50 mM NaCl, 5 mM EDTA, 5 mM EGTA, 2 mM Na₃ VO₄, 10 mM HEPES pH 7.4, 1% Triton X-100, 1% protease inhibitor, 0.5 mM phenylmethylsulfonyl fluoride (PMSF), and 10 mg/mL leupeptin. Protein quantification was performed using a BCA protein assay kit (Pierce, Thermo Fisher Scientific, Inc., Waltham, MA).

### Cell Proliferation Assay

HUVECs were seeded in 96-well plates (Corning, Inc., Corning, NY) at a density of 5,000 cells/well, treated with supplement-free growth media, normal growth media or PEP, followed by staining with INCUCYTE NUCLIGHT Rapid Red (1:500, Essen BioScience, Inc., Ann Arbor, MI). Stained cell plates were placed in the INCUCYTE S3 live-cell analysis system (Essen BioScience, Inc., Ann Arbor, MI) and scanned every six hours. Fluorescent objects were quantified using the INCUCYTE integrated analysis software (Essen BioScience, Inc., Ann Arbor, MI) to calculate proliferation rate.

### Intracellular Staining, Antibodies, and Flow Cytometry

HUVECs were washed twice with PBS, stained with zombie dye (BioLegend, Inc., San Diego, CA), fixed (PERM FIX, BioLegend, Inc., San Diego, CA), washed twice with permeabilization buffer (eBioscience, San Diego, CA), stained with anti-CD31 antibodies (744361, BD Biosciences, Franklin Lakes, NJ) and anti-Ki-67 (11-5698-82, ThermoFisher Scientific, Inc., Waltham, MA) for at least 30 minutes at room temperature. Cells were washed twice with permeabilization buffer (eBioscience, San Diego, CA) before flow cytometry acquisition. Staining antibodies were diluted 1:100 prior to staining. Flow cytometry experiments were performed through an 11-color system (ATTUNE NXT, Life Technologies Corp., Thermo Fisher Scientific, Inc., Carlsbad, CA). Data were then analyzed by FlowJo software (BD Biosciences, Franklin Lakes, NJ).

### Cell Migration Assay

HUVECs were seeded in 96-well plates (Corning, Inc., Corning, NY). Cells were grown to confluency, followed by scratching on cell monolayer using a wound maker (Essen BioScience, Inc., Ann Arbor, MI). After two PBS washes and addition of PEP (2.5×10¹¹ particles/mL) with serum-free medium, cell migration was measured and analyzed using a live cell imaging system (INCUCYTE S3, Essen BioScience, Inc., Ann Arbor, MI).

### Matrigel Tube Formation Assay

96-well plates (Corning, Inc., Corning, NY) were pre-coated with MATRIGEL (Corning Life Sciences, Corning, NY) and allowed to solidify for one hour at 37° C. before cell seeding. GFP-tagged HUVECs (1×10⁴ cells/well) were then added to individual wells in medium designated treatment. Images were acquired at time 0, one hour, three hours, and six hours post-treatment with an inverted microscope (DMI6000 B, Leica Microsystems GmbH, Wetzlar, Germany). All images were analyzed with Angiotool (Zudaire et al., 2011, PLoS One 6:e27385).

### PKH26 Vesicle Labeling

PEP vesicles were labeled with PKH26 red fluorescent dye (MilliporeSigma, Burlington, MA), according to the manufacturer's protocol. Briefly, PEP was resuspended in 1 mL Diluent C, mixed with 4 μL PKH26, and incubated for five minutes at room temperature. Labeling was quenched by addition of 2 mL 10% BSA and 8.5 mL serum-free medium (Lonza Group AG, Basel, Switzerland). Labeled exosomes were ultracentrifuged at 190,000×g for two hours, washed with PBS, and concentrated by centrifugation at 3000×g with a 10 kDa filter column (AMICON, Merck KGaA, Darmstadt, Germany).

### Endocytosis Inhibition Assay

HUVECs were cultured in two-well chamber slides (NUNC LAB-TEK II, Thermo Fisher Scientific, Inc., Waltham, MA) at a density of 150,000 cells/well in EBM-2 basal medium (Lonza Group AG, Basel, Switzerland). Inhibitors heparin, amiloride, dynasore, Pitstop 2, or omeprazole were used to pre-treated cells for 30 minutes before labeled PEP was added to HUVECs, then incubated for six hours at 37° C. Subsequently, medium was discarded, and cells were washed with PBS to remove excess exosomes. Cells were fixed in 4% (vol/vol) paraformaldehyde, permeabilized with 0.5% Triton X-100 in PBS, blocked (blocking buffer: 5% normal donkey serum, 0.2% Triton-X 100 in PBS) and stained with ALEXA FLUOR (Molecular Probes, Inc., Eugene, OR) 488 Phalloidin (Thermo Fisher Scientific, Inc., Waltham, MA). Fluorescent images were obtained using a confocal microscope (LSM 780, Carl Zeiss AG, Oberkochen, Germany). Microscope images were exported as tiff files using Zen Blue and analyzed using ImageJ software (Schneider et al., 2012, Nature Methods 9(7): 671-675).

### Live-Cell Internalization Imaging

To monitor the internalization fate of PEP, HUVECs were cultured with PKH26-labeled PEP for 18 hours. Cells were analyzed using a confocal microscope (LSM 780, Carl Zeiss AG, Oberkochen, Germany) as previously described (Schott et al., 2019, J Cell Biol 218:3320-3335).

### Rat Hind Limb Ischemia (HLI) Model

Rat studies were conducted with 11 male Sprague Dawley (SD) rats ranging in weight from 220-265 g. Animals were randomly divided into three groups as follows: sham group (n=4), TISSEEL group (n=3), and TISSEEL-PEP group (n=4).

During both the surgical procedure and SPY intraoperative laser angiography (SPY, Stryker Corp., Kalamazoo, MI), animals were anesthetized with 1-3% isoflurane and body temperature was maintained on a circulating heated water pad. Following skin incision, the right femoral nerve, artery, and vein were visualized under a stereoscope. Unilateral ischemia damage on the right limb was achieved by ligation and extraction of femoral artery sections as described previously (Mirabella et al., 2017, Nat Biomed Eng 1:0083). Once the artery was occluded, the surgical site was sutured and perfusion of the operated limb was assessed immediately post-surgery via SPY. Rats underwent intramuscular (IM) injection of 0.3 mL saline (Sham), 0.3 mL TISSEEL (Baxter International, Inc., Deerfield, IL), or 0.3 mL TISSEL-PEP in the surgical tissue. The surgical site was inspected for infection for the duration of the study. In this moderate ischemia model, perfusion assessment was performed as described previously, prior to surgery and at 10 minutes and 21 days after surgery (Mücke et al., 2020, Sci Rep. 10:939). Animals had no spontaneous tissue necrosis or self-amputation during the study.

### Rabbit Ear Ischemic Wound Model

Rabbit studies were done with 12 female New Zealand white rabbits ranging in weight from 2.0 kg to 3.5 kg, randomly assigned into four groups as follows: sham group (n=3), TISSEEL group (n=3), PEP group (n=3) and TISSEEL-PEP group (n=3).

During the surgical procedure and SPY intraoperative laser angiography (SPY, Stryker Corp., Kalamazoo, MI), animals were anesthetized with ketamine (35-40 mg/kg) and xylazine (5 mg/kg) and maintained with 2.5-3.5% isoflurane. Body temperature was maintained on a heating pad. The ischemic damage on the ear was done as previously described (Chen, S., 2007, Wound Rep Reg. 15:928-935). The central and cranial arteries were ligated and accompanying nerves cut. Circumferential subcutaneous tunnel was made through the three incisions and all subcutaneous tissues, muscles, nerves, and small vascular branches were resected. Circular full-thickness skin wounds were created on the ventral side of each ear with an 8-mm stainless steel punch. Perfusion of the operated ear was assessed via angiography imaging (SPY elite fluorescence, Stryker Corp., Kalamazoo, MI) immediately prior to surgery and post-surgery at 10 minutes and 28 days. This wound model developed spontaneous tissue necrosis during the study. Wound closure analysis was done with ImageJ software (Schneider et al., 2012, Nature Methods 9(7): 671-675).

### Histology

Rats were sacrificed at 21 days post-surgery. Excised muscle was fixed in 4% (vol/vol) paraformaldehyde, paraffin-embedded, and sectioned at 10 μm thickness.

Rabbits were sacrificed at four weeks post-surgery, and ear skin containing either healthy or wounded skin were fixed in 10% neutral formalin, rinsed in 30% sucrose and 0.1% sodium azide, paraffin-embedded and sectioned at 5 μm thickness.

Hematoxylin and eosin (H&E) staining and Masson's trichrome staining were performed according to standard procedures. To quantify percentages of vascular area, NIS-ELEMENTS (Nikon Instruments, Inc., Melville, NY) software was used to record images. ImageJ software (Schneider et al., 2012, Nature Methods 9(7): 671-675) was used for image analysis.

### Immunohistochemical Analysis

Immunohistochemistry was performed on de-paraffinized sections. Antigens were retrieved using an acid-based antigen unmasking solution (R&D Systems, Inc., Minneapolis, MN). Tissue sections were then permeabilized with 0.5% Triton X-100 in PBS, and blocked (5% normal donkey serum, 0.2% Triton-X 100 in PBS) before incubating in primary antibody at 4° C. overnight with the following antibodies diluted in blocking buffer: anti-vWF (1:400, ab6994, Abcam, Cambridge, United Kingdom), anti-CD31(1:400, NB6300-562, Novus Biologicals, LLC, Centennial, CO) and anti-α-SMA (1:400, NB300-978, Novus Biologicals, LLC, Centennial, CO). Samples were then stained for one hour at room temperature with fluorescent secondary antibodies (Thermo Fisher Scientific Inc., Waltham, MA). For EdU staining, an EdU cell proliferation kit (CLICK-IT Plus, Thermo Fisher Scientific Inc., Waltham, MA) was used per manufacturer's protocol. Slides were mounted with Antifade Mounting Medium with DAPI (Vector Laboratories, Inc., Burlingame, CA) to visualize nuclei, and captured with a confocal microscope (LSM 780, Carl Zeiss AG, Oberkochen, Germany). Microscope images were exported as tiff files using Zen Blue and analyzed using ImageJ software (Schneider et al., 2012, Nature Methods 9(7): 671-675).

### Proteome Profiler Array

To measure PEP-induced angiogenic factors in vitro, HUVEC lysates with or without PEP treatment were analyzed using a human angiogenesis array kit (ARY007, R&D Systems Inc., Minneapolis, MN) according to the manufacturer's instructions. Blots were analyzed with Quick Spots tool from HLImage++ software (Western Vision Software, Salt Lake City, Utah).

Heatmap visualization was conducted by pheatmap (V1.0.12; Luo et al., 2013, Bioinformatics 29:1830-1831).

### Angiogenesis Rt² PCR Array

Total RNA was extracted from muscle samples using RNEASY Fibrous Tissue Kit (Qiagen, Hilden Germany). A total of 0.5 μg RNA was used for cDNA synthesis with an RT² First Strand Kit (Qiagen, Hilden Germany). Angiogenesis RT² Profiler PCR array (Qiagen, Hilden Germany) was designated to detect expression of angiogenesis related genes. Procedures were conducted according to the manufacturer's instructions. Data were analyzed through the online Gene Globe Data Analysis Center (Qiagen, Hilden Germany). For consensus clustering, analysis and heatmap generation were conducted using Morpheus software (Broad Institute, Cambridge, MA).

### Statistical Analysis

Data are presented as mean±S.D. unless otherwise noted, and a p value of less than 0.05 was considered significant. For in vitro studies, Student's t-test, one-way ANOVA followed by Dunnett's post-hoc test or Mann-Whitney test were used where appropriate. For in vivo studies, data were analyzed in investigator-blinded fashion. Two-way ANOVA, nonparametric Mann-Whitney U test or Fisher's exact test were used where appropriate.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of treating peripheral vascular disease, a vascular defect, or vascular dysfunction in a subject, the method comprising administering to the subject a therapeutic composition comprising:
a purified exosome product (PEP); and
a pharmaceutically acceptable carrier.

2. The method of claim 1, wherein the PEP comprises spherical or spheroid exosomes having a diameter no greater than 300 nm.

3. The method of claim 1, wherein the PEP comprises spherical or spheroid exosomes having a mean diameter of 110 nm±90 nm.

4. The method of claim 3, wherein the PEP comprises spherical or spheroid exosomes having a mean diameter of 110 nm±50 nm.

5. The method of claim 4, wherein the PEP comprises spherical or spheroid exosomes having a mean diameter of 110 nm±30 nm.

6. The method of claim 1, wherein the PEP comprises:
from 1% to 20% CD63⁻ exosomes; and
from 80% to 99% CD63⁺ exosomes.

7. The method of claim wherein the PEP comprises at least 50% CD63⁻ exosomes.

8. The method claim 1, wherein the PEP comprises from 1×10¹¹ PEP exosomes to 1×10¹³ PEP exosomes.

9. The method of claim 8, wherein the PEP comprises from 1×10¹² PEP exosomes to 1×10¹³ PEP exosomes.

10. The method claim 1, wherein the therapeutic composition further comprises a supportive matrix.

11. The method of claim 10, wherein the supportive matrix comprises a collagen scaffold.

12. The method claim 1, wherein the therapeutic composition further comprises a tissue sealant, fibrin glue, or a hydrogel.

13. The method claim 1, wherein the therapeutic composition is applied in an amount effective to increase pro-angiogenic activity compared to peripheral vascular disease treated without the therapeutic composition.

14. The method claim 1, wherein the therapeutic composition is applied in an amount effective to increase perfusion in vivo following ischemia compared to peripheral vascular disease treated without the therapeutic composition.

15. The method claim 1, wherein the therapeutic composition is applied in an amount effective to increase drive of the MAPK pathway or the AKT pathway compared to peripheral vascular disease treated without the therapeutic composition.

16. The method claim 1, wherein the therapeutic composition is delivered by intramuscular injection.

17. The method claim 1, wherein the peripheral vascular disease, a vascular defect, or vascular dysfunction comprises peripheral arterial disease.

18. The method claim 1, wherein the peripheral vascular disease, a vascular defect, or vascular dysfunction comprises atherosclerosis, ischemia, deep vein thrombosis, pulmonary embolism, varicose veins, chromic venous insufficiency, Buerger disease, Reynaud phenomenon, thrombophlebitis, or an aneurysm.

19. The method claim 1, wherein the subject is a human.

20. The method claim 1, wherein the subject has an ischemic wound, and wherein the method increases ischemic wound closure as compared to a method not including PEP.
